(19) United States
(12) Patent Application Publication
(10) Pub. No.: US 2026/0127814 A1
(43) Pub. Date: May 7, 2026

(54) SYSTEM FOR ENHANCING 3D SEGMENTATION COMPREHENSION THROUGH DYNAMIC 4D ANATOMICAL MARKERS

(71) Applicant: GE Precision Healthcare LLC, Waukesha, WI (US)

(72) Inventors: Hani Nozari MIRAR, Oslo (NO); Alf GRINI, Oslo (NO)

(21) Appl. No.: 18/939,716

(22) Filed: Nov. 7, 2024

(51) Int. Cl. G06T 17/00; A61B 8/00; G06T 7/00; G06T 7/11; G06T 7/73; G06T 19/20

(52) U.S. Cl. CPC G06T 17/00; A61B 8/4494; A61B 8/461; G06T 7/0012; G06T 7/11; G06T 7/75; G06T 19/20; G06T 2200/08; G06T 2200/24; G06T 2207/10132; G06T 2207/30004; G06T 2207/30204; G06T 2210/41; G06T 2219/2016

(57) ABSTRACT

A ultrasound imaging system may include a transducer configured to transmit and receive ultrasound signals; a matching layer configured to have an acoustic impedance between a tissue to be imaged and a material of the transducer; a damping block configured to absorb ultrasound energy; and a processing circuit. The processing circuit may acquire medical imaging data of an anatomical feature of a subject. The processing circuit may determine a position of an anatomical structure in relation to the anatomical feature of the subject. The processing circuit may generate a four-dimensional (4D) model of the anatomical feature of the subject that includes a visual indicator that identifies the position of the anatomical structure in relation to the anatomical feature of the subject. The processing circuit may display the 4D model via a user interface.

### TECHNICAL FIELD

The present disclosure relates to a system for generating a four-dimensional (4D) model of an anatomical feature of a subject that includes a visual indicator that identifies the position of an anatomical structure in relation to the anatomical feature of the subject.

### BACKGROUND

A medical imaging system may acquire medical images of an anatomical feature in a region of interest of a subject. Further, the medical imaging system may segment the anatomical feature in the medical images, and generate a model of the anatomical feature. The medical imaging system may display the model of the anatomical feature via a user interface to permit a user to view and assess the anatomical feature and gauge the precision of the segmentation. In some cases, users that lack experience or have a limited understanding of anatomy may find it challenging to interpret the displayed model. For instance, the users may find it difficult to assess the positions of various anatomical structures of the anatomical feature, and/or assess the quality and precision of the segmentation of the anatomical feature. This problem may be exacerbated in situations where the displayed model is a 4D model because the model is moving with time.

### SUMMARY

This summary introduces concepts that are described in more detail in the detailed description. It should not be used to identify essential features of the claimed subject matter, nor to limit the scope of the claimed subject matter.

In an aspect, a system may include a memory configured to store instructions; and one or more processors configured to execute the instructions to: acquire medical imaging data of an anatomical feature of a subject; determine a position of an anatomical structure in relation to the anatomical feature of the subject; generate a four-dimensional (4D) model of the anatomical feature of the subject that includes a visual indicator that identifies the position of the anatomical structure in relation to the anatomical feature of the subject; and display the 4D model via a user interface.

In another aspect, a method may include acquiring medical imaging data of an anatomical feature of a subject; determining a position of an anatomical structure in relation to the anatomical feature of the subject; generating a four-dimensional (4D) model of the anatomical feature of the subject that includes a visual indicator that identifies the position of the anatomical structure in relation to the anatomical feature of the subject; and displaying the 4D model via a user interface.

In yet another aspect, a non-transitory computer-readable medium may store instructions that, when executed by one or more processors, cause the one or more processors to: acquire medical imaging data of an anatomical feature of a subject; determine a position of an anatomical structure in relation to the anatomical feature of the subject; generate a four-dimensional (4D) model of the anatomical feature of the subject that includes a visual indicator that identifies the position of the anatomical structure in relation to the anatomical feature of the subject; and display the 4D model via a user interface.

### BRIEF DESCRIPTION OF DRAWINGS

FIG. 1 is a diagram of an example system for generating a 4D model of an anatomical feature of a subject that includes a visual indicator that identifies a position of an anatomical structure in relation to the anatomical feature of the subject.

FIG. 2 is a diagram of example components of one or more devices of FIG. 1.

FIG. 3 is a diagram of an example medical imaging system.

FIG. 4 is a diagram of an example medical imaging system.

FIG. 5 is a flowchart of an example process for generating a 4D model of an anatomical feature of a subject that includes a visual indicator that identifies a position of an anatomical structure in relation to the anatomical feature of the subject.

FIGS. 6A and 6B are diagrams of an example user interface for displaying a 4D model of an anatomical feature of a subject that includes a visual indicator that identifies a position of an anatomical structure in relation to the anatomical feature of the subject.

FIG. 7 is a flowchart of an example process for adjusting a displayed 4D model based on a user input while maintaining a position of a visual indicator relative to the displayed 4D model.

FIGS. 8A and 8B are diagrams of an example user interface for displaying a 4D model that is adjusted based on a user input while maintaining a position of a visual indicator relative to the displayed 4D model.

FIG. 9 is a flowchart of an example process for adjusting an image parameter of a second visual indicator that identifies an anatomical structure based on a distance between a first visual indicator that identifies a position of the anatomical structure and a viewing plane of a displayed 4D model.

FIGS. 10A-10C are diagrams of an example user interface for displaying and adjusting an image parameter of a second visual indicator that identifies an anatomical feature based on a distance between a first visual indicator, that identifies a position of the anatomical feature in relation to an anatomical feature, and a viewing plane of a displayed 4D model.

FIG. 11 is flowchart of an example process for setting an initial view of a displayed 4D model to depict a region of the displayed 4D model having a segmentation quality that is less than a threshold.

FIG. 12 is a diagram of an example user interface for displaying an initial view of a 4D model to depict a region of the displayed 4D model having a segmentation quality that is less than a threshold.

### DETAILED DESCRIPTION

As addressed above, a medical imaging system may generate a model of an anatomical feature of a subject. Some users might find it difficult to interpret the model and/or gauge the precision of the segmentation of the anatomical feature of the subject. Further, these problems may be exacerbated in situations where the model is a 4D model because the model changes over time.

Some embodiments herein provide an ultrasound imaging system that includes a transducer configured to transmit and receive ultrasound signals; a matching layer configured to have an acoustic impedance between a tissue to be imaged and a material of the transducer; a damping block configured to absorb ultrasound energy; and a processing circuit configured to acquire medical imaging data of an anatomical feature of a subject, determine a position of an anatomical structure in relation to the anatomical feature of the subject, generate a 4D model of the anatomical feature of the subject that includes a visual indicator that identifies the position of the anatomical structure in relation to the anatomical feature of the subject, and display the 4D model via a user interface. In this way, the user can view the 4D model and readily assess the position of the anatomical structure in relation to the anatomical feature based on the position of the visual indicator. Further, the ultrasound imaging system may maintain the position of the visual indicator relative to the anatomical feature as the user manipulates the displayed 4D model, which may further improve the user's ability to assess the interrelationship of the anatomical structure and the anatomical feature. Further still, the ultrasound imaging system may display a second visual indicator that identifies the anatomical structure, and adjust an image parameter of the second visual indicator based on the extent of visibility of the anatomical structure in a viewing plane of the 4D model.

In this way, some embodiments herein provide a technical improvement in the technical field of medical imaging by generating and displaying more visually-informative 4D models that are generated based on medical imaging data. Further, in this way, some embodiments herein provide a technical improvement to medical imaging systems by providing an improved user interface that displays more visually-informative 4D models and information that permits a user to readily assess the position of an anatomical structure relative to an anatomical feature and readily assess the extent of visibility of the anatomical structure in a viewing plane of the 4D model.

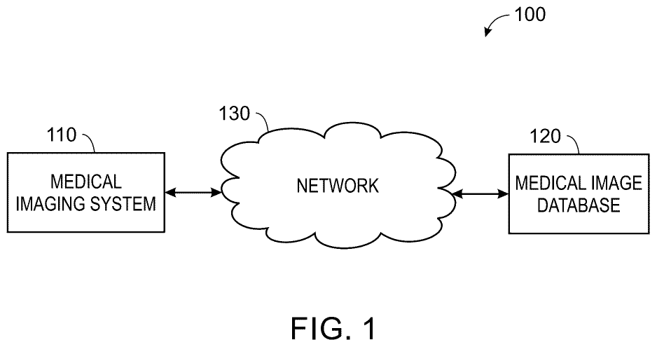

FIG. 1 is a diagram of an example system 100 for generating a 4D model of an anatomical feature of a subject that includes a visual indicator that identifies the position of the anatomical structure in relation to the anatomical feature of the subject. As shown in FIG. 1, the system 100 may include a medical imaging system 110, a medical imaging database 120, and a network 130.

The medical imaging system 110 may be configured to acquire medical imaging data of an anatomical feature of a subject; determine a position of an anatomical structure in relation to the anatomical feature of the subject; generate a four-dimensional (4D) model of the anatomical feature of the subject that includes a visual indicator that identifies the position of the anatomical structure in relation to the anatomical feature of the subject; and display the 4D model via a user interface. For example, the medical imaging system 110 may be a computer, a server, an ultrasound system, a computed tomography (CT) system, a magnetic resonance imaging (MRI) system, an ultrasound system, an X-ray system, a positron emission tomography (PET) device, or the like.

The medical imaging database 120 may be configured to store medical imaging data of an anatomical feature of a subject. For example, the medical imaging database 120 may be a cloud database, a hierarchical database, a network database, a centralized database, a picture archiving and communication system (PACS), or the like.

The network 130 may permit communication between the medical imaging system 110 and the medical imaging database 120. For example, the network 150 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

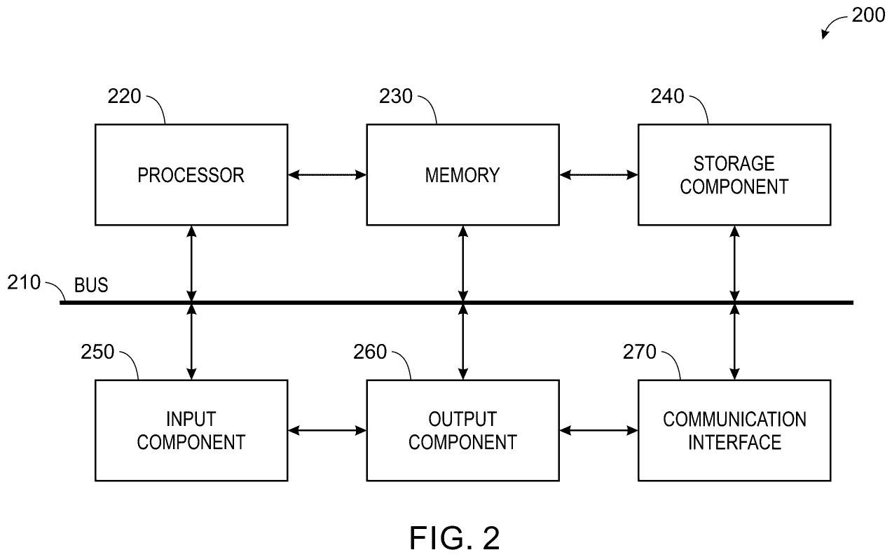

FIG. 2 is a diagram of example components of one or more devices of FIG. 1. The device 200 may correspond to the medical imaging system 110 and/or the medical imaging database 130. As shown in FIG. 2, the device 200 may include a bus 210, a processor 220, a memory 230, a storage component 240, an input component 250, an output component 260, and a communication interface 270.

The bus 210 includes a component that permits communication among the components of the device 200. The processor 220 may be implemented in hardware, firmware, or a combination of hardware and software. The processor 220 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 220 may include one or more processors capable of being programmed to perform a function. The processor 220 may include one or more processors 220 configured to perform the operations described herein. For example, a single processor 220 may be configured to perform all of the operations described herein. Alternatively, multiple processors 220, collectively, may be configured to perform all of the operations described herein, and each of the multiple processors 220 may be configured to perform a subset of the operations descried herein. For example, a first processor 220 may perform a first subset of the operations described herein, a second processor 220 may be configured to perform a second subset of the operations described herein, etc.

The memory 230 may include a random access memory (RAM), a read only memory (ROM), and/or another type of dynamic or static storage device (e.g., a flash memory, a magnetic memory, and/or an optical memory) that stores information and/or instructions for use by the processor 220.

The storage component 240 may store information and/or software related to the operation and use of the device 200. For example, the storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 may include a component that permits the device 200 to receive information, such as via user input (e.g., a touch screen display, a keyboard, a keypad, a mouse, a button, a switch, a camera, and/or a microphone). Additionally, or alternatively, the input component 250 may include a sensor for sensing information (e.g., a global positioning system (GPS) component, an accelerometer, a gyroscope, and/or an actuator). The output component 260 may include a component that provides output information from the device 200 (e.g., a display, a speaker for outputting sound at the output sound level, and/or one or more light-emitting diodes (LEDs)).

The communication interface 270 may include a transceiver-like component (e.g., a transceiver and/or a separate receiver and transmitter) that enables the device 200 to communicate with other systems, such as via a wired connection, a wireless connection, or a combination of wired and wireless connections. The communication interface 270 may permit the device 200 to receive information from another system and/or provide information to another system. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The device 200 may perform one or more processes described herein. The device 200 may perform these processes based on the processor 220 executing software instructions stored by a non-transitory computer-readable medium, such as the memory 230 and/or the storage component 240. A computer-readable medium may be defined herein as a non-transitory memory device. A memory device may include memory space within a single physical storage device or memory space spread across multiple physical storage devices.

The software instructions may be read into the memory 230 and/or the storage component 240 from another computer-readable medium or from another system via the communication interface 270. When executed, the software instructions stored in the memory 230 and/or the storage component 240 may cause the processor 220 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components of the device 200 shown in FIG. 2 are provided as an example. In practice, the device 200 may include additional components, fewer components, different components, or differently arranged components than those shown in FIG. 2. Additionally, or alternatively, a set of components (e.g., one or more components) of the device 200 may perform one or more functions described as being performed by another set of components of the device 200.

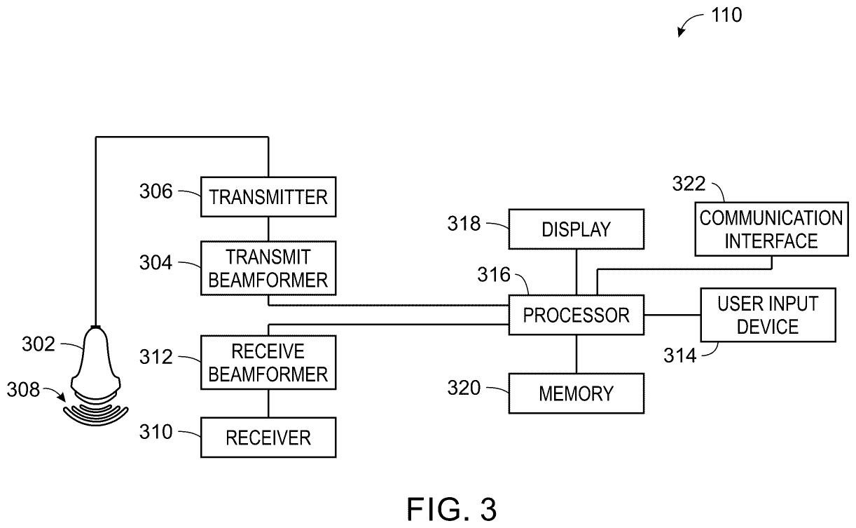

FIG. 3 is a diagram of an example medical imaging system 110. As shown in FIG. 3, the medical imaging system 110 may include an ultrasound probe 302, a transmit beamformer 304, a transmitter 306, elements 308 a receiver 310, a receive beamformer 312, a user input device 314, a processor 316, a display 318, a memory 320, and a communication interface 322. The foregoing components may be connected via wired or wireless connections.

The ultrasound probe 302 may be configured to acquire ultrasound data of a region of interest of a subject. For example, the ultrasound probe 302 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 302 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data. The ultrasound probe 302 may include a transducer configured to transmit and receive ultrasound signals, a matching layer configured to have an acoustic impedance between a tissue to be imaged and a material of the transducer, and a damping block configured to absorb ultrasound energy.

The transmit beamformer 304 may be configured to apply delay times to electrical signals provided to the elements 308 to focus corresponding ultrasound signals at the region of interest. The transmitter 306 may be configured to transmit electrical signals to the elements 308 to drive the elements 308 to emit ultrasound signals towards the region of interest. The elements 308 may be configured to receive the electrical signals from the transmitter 306, convert the electrical signals into ultrasound signals, and emit the ultrasound signals towards the region of interest. The elements 308 may be configured to receive echo ultrasound signals that are back-scattered by the region of interest, convert the echo ultrasound signals into electrical signals, and provide the electrical signals to the receiver 310. The receiver 310 may be configured to receive electrical signals from the elements 308, and provide the electrical signals to the receive beamformer 312. The receive beamformer 312 may apply delay times to the electrical signals received from the elements 308.

The user input device 314 may be configured to receive a user input, and provide the user input to the processor 316. For example, the user input device 314 may be a touch screen display, a keyboard, a keypad, a mouse, a button, a switch, a microphone, or the like. Additionally, or alternatively, the user input device 314 may be configured to sense information. For example, the user input device 314 may sense information from an electro-magnetic positioning system, an inertial measurement system, an accelerometer, a gyroscope, an actuator, or the like.

The processor 316 may be configured to perform the operations as described herein. For example, the processor 316 may be a processing circuit, a CPU, a GPU, an APU, a microprocessor, a microcontroller, a DSP, an FPGA, an ASIC, or another type of processing component. The processor 316 may be implemented in hardware, firmware, or a combination of hardware and software. The processor 316 may include one or more processors 316 configured to perform the operations described herein. For example, a single processor 316 may be configured to perform all of the operations described herein. Alternatively, multiple processors 316, collectively, may be configured to perform all of the operations described herein, and each of the multiple processors 316 may be configured to perform a subset of the operations descried herein. For example, a first processor 316 may perform a first subset of the operations described herein, a second processor 316 may be configured to perform a second subset of the operations described herein, etc.

The processor 316 may be configured to control the ultrasound probe 302 to acquire ultrasound data. The processor 316 may be configured to control which of the elements 308 are active, and control the shape of a beam emitted from the ultrasound probe 302. The processor 316 may generate ultrasound images for display. For example, the processor 316 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 318 may be configured to display information. For example, the display 318 may be a monitor, an LED display, a cathode ray tube, a projector display, a touchscreen, tablet computer, mobile phone, or the like. The display 318 may display ultrasound images based on the ultrasound data in real-time. For example, the display 318 may display the ultrasound images within one second, two seconds, five seconds, etc., of the ultrasound data being acquired by the ultrasound probe 302.

The memory 320 may be configured to store information and/or instructions for use by the processor 316. The memory 320 may be a non-transitory computer-readable medium. For example, the memory 320 may be a RAM, a ROM, and/or another type of dynamic or static storage device (e.g., a flash memory, a magnetic memory, and/or an optical memory) that stores information and/or instructions for use by the processor 316. The memory 320 may be configured to store instructions that, when executed by the processor 316, cause the processor 316 to perform the operations described herein.

The communication interface 322 may be configured to enable the processor 316 to communicate with other systems, such as via a wired connection, a wireless connection, or a combination of wired and wireless connections. For example, the communication interface 322 may include an Ethernet interface, an optical interface, a coaxial interface, an infrared interface, an RF interface, a USB interface, a Wi-Fi interface, a cellular network interface, or the like.

The number and arrangement of the components of the medical imaging system 110 shown in FIG. 3 are provided as an example. In practice, the medical imaging system 110 may include additional components, fewer components, different components, or differently arranged components than those shown in FIG. 3. Additionally, or alternatively, a set of components (e.g., one or more components) of the medical imaging system 110 may perform one or more functions described as being performed by another set of components of the medical imaging system 110.

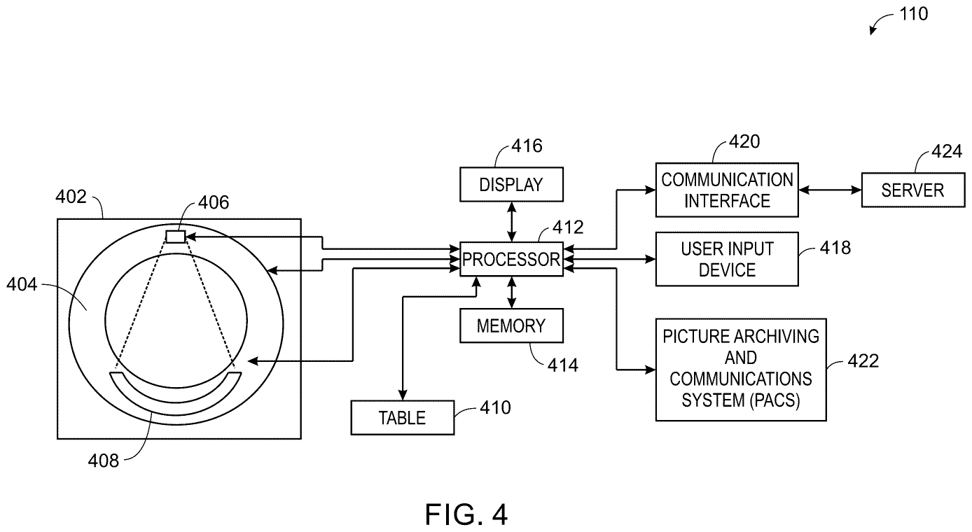

FIG. 4 is a diagram of an example medical imaging system 110. As shown in FIG. 4, the medical imaging system 110 may include a gantry 402, a rotational frame 404, an X-ray source 406, an X-ray detector 408, a table 410, a processor 412, a memory 414, a display 416, a user input device 418, a communication interface 420, a PACS 422, and a server 424.

The processor 412 may be configured to control operations of the preoperative imaging system 130. For example, the processor 412 may be a CPU, a GPU, an APU, a microprocessor, a microcontroller, a DSP, an FPGA, an ASIC, or the like. The processor 412 may be implemented in hardware, firmware, or a combination of hardware and software. The processor 412 may include one or more processors 412 configured to perform the operations described herein. For example, a single processor 412 may be configured to perform all of the operations described herein. Alternatively, multiple processors 412, collectively, may be configured to perform all of the operations described herein, and each of the multiple processors 412 may be configured to perform a subset of the operations descried herein. For example, a first processor 412 may perform a first subset of the operations described herein, a second processor 412 may be configured to perform a second subset of the operations described herein, etc.

The processor 412 may be configured to control the gantry 402, movement of the rotational frame 404, the X-ray source 406, the X-ray detector 408, and movement of the table 410.

The memory 414 may be configured to store information and/or instructions for use by the processor 412. The memory 414 may be a non-transitory computer-readable medium. For example, the memory 414 may be a RAM, a ROM, a flash memory, a magnetic memory, an optical memory, or the like. The memory 414 may be configured to store instructions that, when executed by the processor 412, cause the processor 412 to perform the operations described herein.

The display 416 may be configured to display information. For example, the display 416 may be a monitor, an LED display, a cathode ray tube, a projector display, a touchscreen, tablet computer, mobile phone, or the like.

The user input device 418 may be configured to receive a user input, and provide the user input to the processor 412. For example, the user input device 418 may be a touch screen display, a keyboard, a keypad, a mouse, a button, a switch, a microphone, or the like. Additionally, or alternatively, the user input device 418 may be configured to sense information. For example, the user input device 418 may sense information from an electro-magnetic positioning system, an inertial measurement system, an accelerometer, a gyroscope, an actuator, or the like.

The communication interface 420 may be configured to enable the processor 412 to communicate with other devices, such as via a wired connection, a wireless connection, or a combination of wired and wireless connections. For example, the communication interface 420 may include an Ethernet interface, an optical interface, a coaxial interface, an infrared interface, an RF interface, a USB interface, a Wi-Fi interface, a cellular network interface, or the like. The PACS 422 may be configured to communicate with external systems and/or networks to permit users at various locations to access the medical image.

The number and arrangement of the components of the medical imaging system 110 shown in FIG. 4 are provided as an example. In practice, the medical imaging system 110 may include additional components, fewer components, different components, or differently arranged components than those shown in FIG. 4. Additionally, or alternatively, a set of components (e.g., one or more components) of the medical imaging system 110 may perform one or more functions described as being performed by another set of components of the medical imaging system 110.

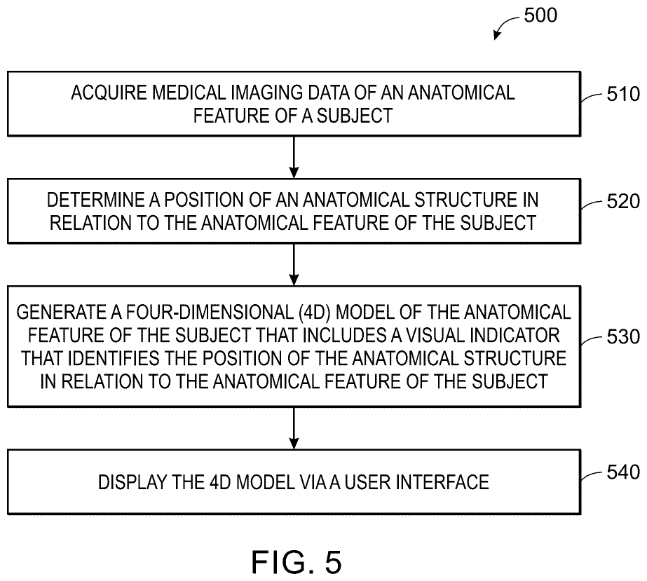

FIG. 5 is a flowchart of an example process 500 for generating a 4D model of an anatomical feature of a subject that includes a visual indicator that identifies the position of an anatomical structure in relation to the anatomical feature of the subject. According to an embodiment, the process 500 may be performed by the medical imaging system 110. Alternatively, one or more operations of the process 500 may be performed by another device.

As shown in FIG. 5, the process 500 may include acquiring medical imaging data of an anatomical feature of a subject (operation 510). For example, the medical imaging system 110 may acquire medical imaging data of an anatomical feature of a subject. The medical imaging data may be ultrasound data, CT data, MRI data, X-ray data, PET data, or the like. The anatomical feature may be any anatomical feature of a subject. For example, the anatomical feature may be a heart, a liver, a brain, or the like. The subject may be a patient, an animal, a phantom, or the like. The medical imaging system 110 may acquire the medical imaging data by performing a scan of the subject. Alternatively, the medical imaging system 110 may acquire the medical imaging data from the medical imaging database 120. In this case, the medical imaging system 110 may have previously acquired the medical imaging data of the subject, or another device may have performed a scan to acquire the medical imaging data of the subject.

As further shown in FIG. 5, the process 500 may include determining a position of an anatomical structure in relation to the anatomical feature of the subject (operation 520). For example, the medical imaging system 110 may determine a position of an anatomical structure in relation to the anatomical feature of the subject. The anatomical structure may be any anatomical structure. The anatomical structure may be an anatomical structure of the anatomical feature, or may be an anatomical structure that is located in proximity to the anatomical feature. As an example, if the anatomical feature is the left atrium and/or the left ventricle of the heart, then the anatomical structure may be the left ventricular outflow tract, the coronary sinus, the pulmonary veins, the left atrial appendage, or the like. As another example, if the anatomical feature is the right atrium and/or the right ventricle of the heart, then the anatomical feature may be an atrial appendage, a coronary sinus, or the like. Although some embodiments herein describe the anatomical feature as being a cardiac feature, and the anatomical structures as being cardiac structures, it should be understood that the embodiments herein are applicable to any anatomical region. For instance, as another example, if the anatomical feature is the liver, then the anatomical structure may be a left lobe, a right lobe, a coronary ligament, a left triangular ligament, the gallbladder, or the like. As yet another example, if the anatomical feature is the brain, then the anatomical structure may be the hypothalamus, the spinal cord, the pineal gland, the medulla oblongata, or the like.

According to an embodiment, the medical imaging system 110 may segment the anatomical feature in the medical imaging data. For example, the medical imaging system 110 may segment the anatomical feature using a segmentation technique (e.g., an artificial intelligence (AI) model, object detection, edge detection, or the like). Further, the medical imaging system 110 may segment the anatomical structure using a segmentation technique. The medical imaging system 110 may determine the position of the anatomical structure in relation to the anatomical feature of the subject based on segmenting the anatomical feature and the anatomical structure.

As further shown in FIG. 5, the process 500 may include generating a four-dimensional (4D) model of the anatomical feature of the subject that includes a visual indicator that identifies the position of the anatomical structure in relation to the anatomical feature of the subject (operation 530). For example, the medical imaging system 110 may generate a 4D model of the anatomical feature of the subject that includes a visual indicator that identifies the position of the anatomical structure in relation to the anatomical feature of the subject. The 4D model may depict the anatomical feature and the anatomical structure in 3D across a timeframe. Restated, the 4D model may depict the anatomical feature and the anatomical structure in three spatial dimensions and a time dimension. The timeframe may be an amount of time corresponding to the medical imaging data, an amount of time associated with an anatomical event (e.g., a cardiac cycle), or the like. The 4D model may include a visual indicator corresponding to the anatomical structure. For example, the visual indicator may be an icon, a label, a 3D representation, a geometric shape, or the like. The medical imaging system 110 may generate the 4D model such that the position of the visual indicator identifies a position of the anatomical structure in relation to the anatomical feature of the subject. According to an embodiment, the position of the visual indicator may remain substantially stationary relative to the anatomical feature. For example, the position of the corresponding anatomical structure may remain substantially stationary relative to the anatomical feature. Alternatively, the position of the visual indicator may vary relative to the anatomical feature across a timeframe. For example, the position of the corresponding anatomical structure may vary relative to the anatomical feature across a timeframe.

As further shown in FIG. 5, the process 500 may include displaying the 4D model via a user interface (operation 540). For example, the medical imaging system 110 may display the 4D model via a user interface. In this way, the medical imaging system 110 may display the 4D model that depicts the anatomical feature and the visual indicator that identifies the position of the anatomical structure in relation to the anatomical feature.

According to an embodiment, the 4D model may depict a single anatomical feature of the subject that includes a single visual indicator that identifies the position of a single anatomical structure in relation to the single anatomical feature of the subject. For example, the 4D model may depict a single anatomical feature (e.g., left ventricle) of the subject that includes a single visual indicator that identifies the position of a single anatomical structure (e.g., left ventricular outflow tract) in relation to the single anatomical feature of the subject.

According to another embodiment, the 4D model may depict a single anatomical feature of the subject that includes multiple visual indicators that respectively identify the positions of multiple corresponding anatomical structures in relation to the single anatomical feature of the subject. For example, the 4D model may depict a single anatomical feature (e.g., left ventricle) of the subject that includes multiple visual indicators that respectively identify the positions of multiple corresponding anatomical structures (e.g., left ventricular outflow tract, mitral valve, etc.) in relation to the single anatomical feature of the subject.

According to another embodiment, the 4D model may depict multiple anatomical features of the subject. Further, each anatomical feature may include a single visual indicator that identifies the position of a single anatomical structure in relation to the single anatomical feature of the subject. For example, the 4D model may depict multiple anatomical features (e.g., left ventricle, right ventricle, etc.) of the subject. Further, each anatomical feature may include a single visual indicator that identifies the position of a single anatomical structure (e.g., left ventricular outflow tract, coronary sinus, etc.) in relation to the single anatomical feature of the subject.

According to another embodiment, the 4D model may depict multiple anatomical features of the subject. Further, each anatomical feature may include multiple visual indicators that respectively identify the positions of corresponding anatomical structures in relation to the anatomical features of the subject. For example, the 4D model may depict multiple anatomical features (e.g., left ventricle, right ventricle, etc.) of the subject. Further, each anatomical feature may include multiple visual indicators that respectively identify the positions of corresponding anatomical structures (e.g., left ventricular outflow tract, coronary sinus, atrial appendage, etc.) in relation to the anatomical features of the subject.

According to another embodiment, the 4D model may depict multiple anatomical features of the subject. Further, a first anatomical feature may include a single visual indicator that identifies the position of a single anatomical structure in relation to the first anatomical feature of the subject, and a second anatomical feature may include multiple visual indicators that respectively identify the positions of corresponding anatomical structures in relation to the second anatomical feature of the subject. For example, the 4D model may depict a first anatomical feature (e.g., left ventricle) that may include a single visual indicator that identifies the position of a single anatomical structure (e.g., left ventricular outflow tract) in relation to the first anatomical feature of the subject, and depict a second anatomical feature (e.g., right ventricle) that may include multiple visual indicators that respectively identify the positions of corresponding anatomical structures (e.g., coronary sinus, atrial appendage, etc.) in relation to the second anatomical feature of the subject.

In this way, it should be understood that the embodiments herein may depict any number of anatomical features and any number of corresponding anatomical structures.

Although FIG. 5 depicts particular operations and a particular sequence of operations, it should be understood that the process 500 may include different operations or differently sequenced operations than as shown in FIG. 5 in other embodiments.

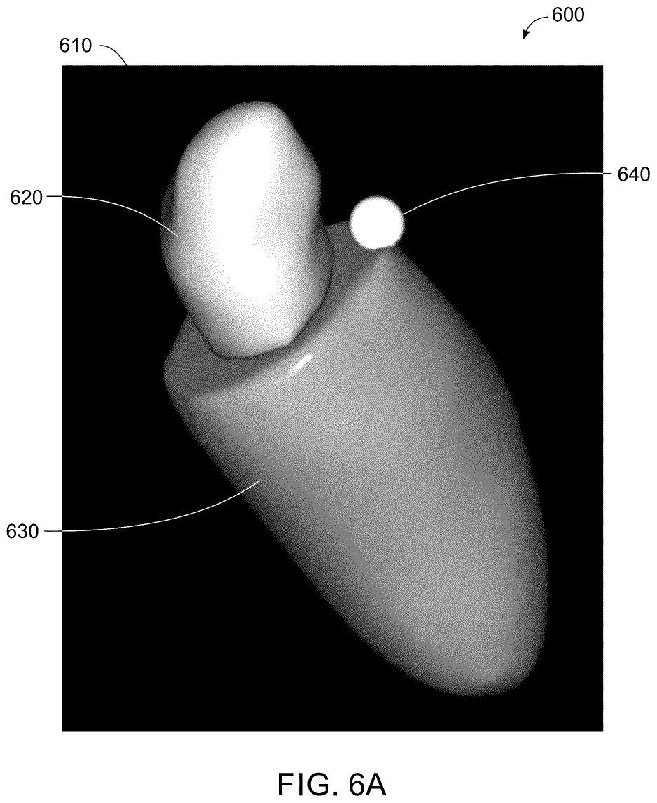

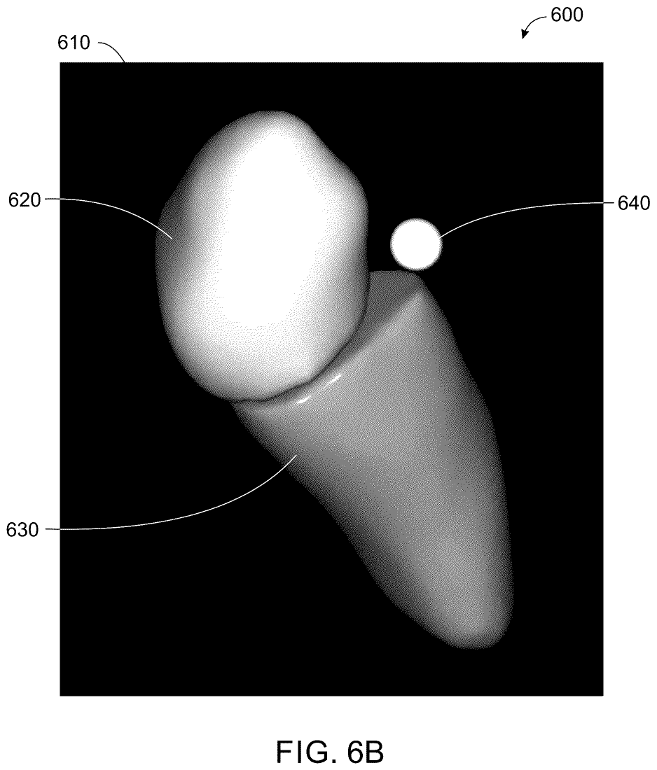

FIGS. 6A and 6B are diagrams of an example user interface 600 for displaying a 4D model of an anatomical feature of a subject that includes a visual indicator that identifies a position of an anatomical structure in relation to the anatomical feature of the subject. As shown in FIG. 6A, the medical imaging system 110 may display a 4D model 610 that displays the left atrium 620 and the left ventricle 630. In this case, the anatomical feature may be the left atrium 620, the left ventricle 630, or the left atrium 620 and the left ventricle 630. Further, as shown in FIG. 6A, the medical imaging system 110 may display a visual indicator 640 that identifies a position of a left ventricular outflow tract. In this case, the anatomical structure may be the left ventricular outflow tract. As shown in FIG. 6B, the 4D model 610 may display the left atrium 620 and the left ventricle 630 across a timeframe such that the left atrium 620 and left ventricle 630 change proportions based on underlying expansion and contraction across the timeframe.

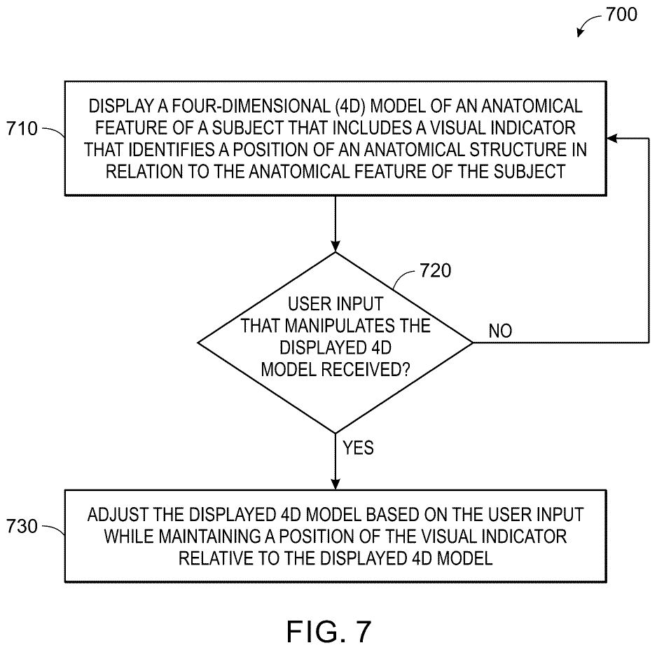

FIG. 7 is a flowchart of an example process 700 for adjusting a displayed 4D model based on a user input while maintaining a position of a visual indicator relative to the displayed 4D model. According to an embodiment, the process 700 may be performed by the medical imaging system 110. Alternatively, one or more operations of the process 700 may be performed by another device.

As shown in FIG. 7, the process 700 may include displaying a four-dimensional (4D) model of an anatomical feature of a subject that includes a visual indicator that identifies a position of an anatomical structure in relation to the anatomical feature of the subject (operation 710). For example, the medical imaging system 110 may display a 4D model of an anatomical feature of a subject that includes a visual indicator that identifies a position of an anatomical structure in relation to the anatomical feature of the subject. For instance, the medical imaging system 110 may display the 4D model in a similar manner as described above in connection with operation 540 of FIG. 5.

As further shown in FIG. 7, the process 700 may include determining whether a user input that manipulates the displayed 4D model is received (operation 720). For example, the medical imaging system 110 may determine whether a user input that manipulates the displayed 4D model is received. The user may interact with the user interface to manipulate the displayed 4D model. For example, the user may zoom in on the 4D model, zoom out of the 4D model, rotate the 4D model about one or more axes, move the 4D model, adjust a focal point of the 4D model, adjust a viewing plane of the 4D model, adjust the 4D model, increase a size of the 4D model, decrease a size of the 4D model, or the like. The medical imaging system 110 may detect a user input, and determine whether the user input is received based on detecting the user input.

As further shown in FIG. 7, if a user input is not received (operation 720-NO), then the process 700 may return to operation 710. For example, the medical imaging system 110 may maintain the display of the 4D model based on determining that a user input is not received.

As further shown in FIG. 7, if a user input that manipulates the displayed 4D model is received (operation 720-YES), then the process 700 may include adjusting the displayed 4D model based on the user input while maintaining a position of the virtual indicator relative to the displayed 4D model (operation 730). For example, the medical imaging system 110 may adjust the displayed 4D model while maintaining a position of the virtual indicator relative to the displayed 4D model based on determining that a user input is received. For instance, the medical imaging system 110 may zoom in on the 4D model, zoom out of the 4D model, rotate the 4D model about one or more axes, move the 4D model, adjust a focal point of the 4D model, adjust a viewing plane of the 4D model, adjust the 4D model, increase a size of the 4D model, decrease a size of the 4D model, or the like, while maintaining a position of the virtual indicator relative to the displayed 4D model.

According to an embodiment, the position of the visual indicator may remain substantially stationary relative to the anatomical feature. For example, the position of the corresponding anatomical structure may remain substantially stationary relative to the anatomical feature. Alternatively, the position of the visual indicator may vary relative to the anatomical feature across a timeframe. For example, the position of the corresponding anatomical structure may vary relative to the anatomical feature across a timeframe. In any event, medical imaging system 110 may adjust the displayed 4D model while maintaining a general position of the virtual indicator relative to the displayed 4D model.

Although FIG. 7 depicts particular operations and a particular sequence of operations, it should be understood that the process 700 may include different operations or differently sequenced operations than as shown in FIG. 7 in other embodiments.

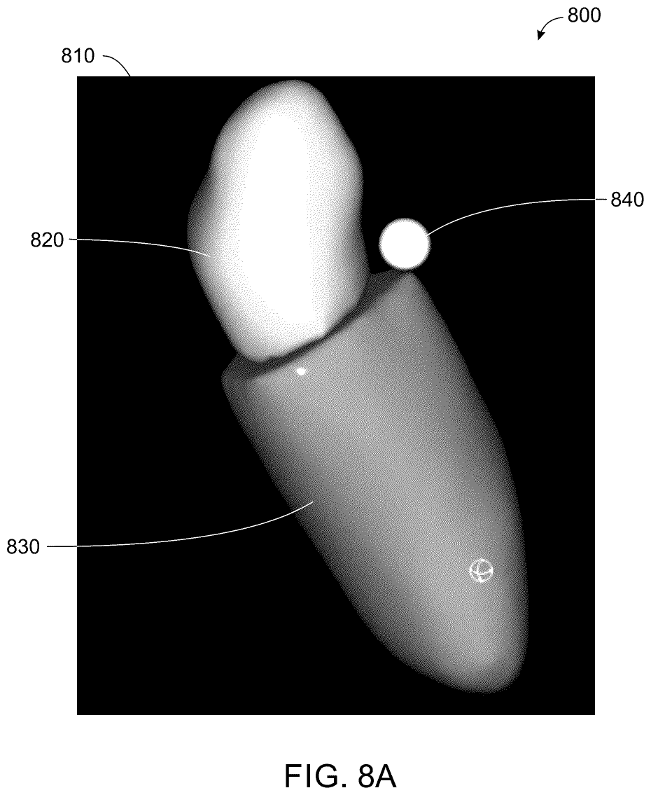

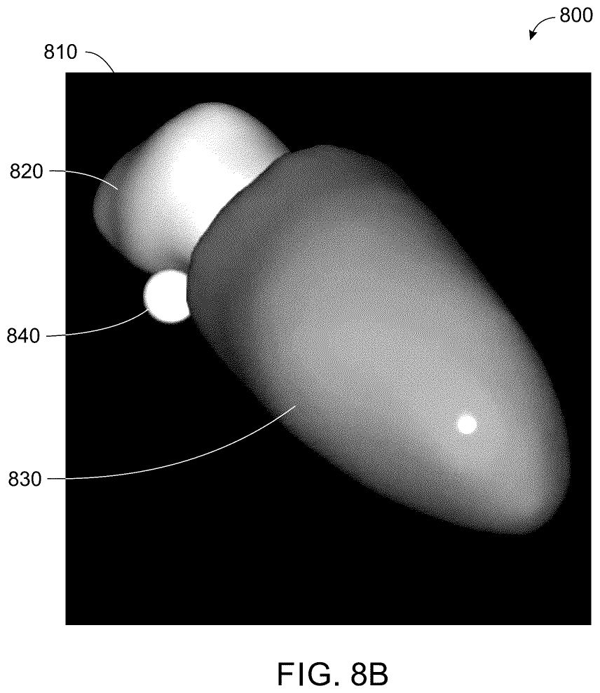

FIGS. 8A and 8B are diagrams of an example user interface 800 for displaying a 4D model that is adjusted based on a user input while maintaining a position of a visual indicator relative to the displayed 4D model. As shown in FIG. 8A, the medical imaging system 110 may display a 4D model 810 that displays the left atrium 820 and the left ventricle 830. In this case, the anatomical feature may be the left atrium 820, the left ventricle 830, or the left atrium 820 and the left ventricle 830. Further, as shown in FIG. 8A, the medical imaging system 110 may display a visual indicator 840 that identifies a position of a left ventricular outflow tract. In this case, the anatomical structure may be the left ventricular outflow tract. As shown in FIG. 8B, the medical imaging system 110 may rotate the displayed 4D model based on a user input that rotates the displayed 4D model. In this case, the positioning of the displayed 4D model may be different than as shown in FIG. 8A because the displayed 4D model has been rotated. However, as shown, the medical imaging system 110 may maintain a position of the visual indicator 840 relative to the displayed 4D model 810. Restated, the position of the visual indicator 840 may move with the underlying 4D model 810 such that the position of the visual indicator 840 relative to the underlying 4D model 810 may be maintained despite movement of the displayed 4D model 810.

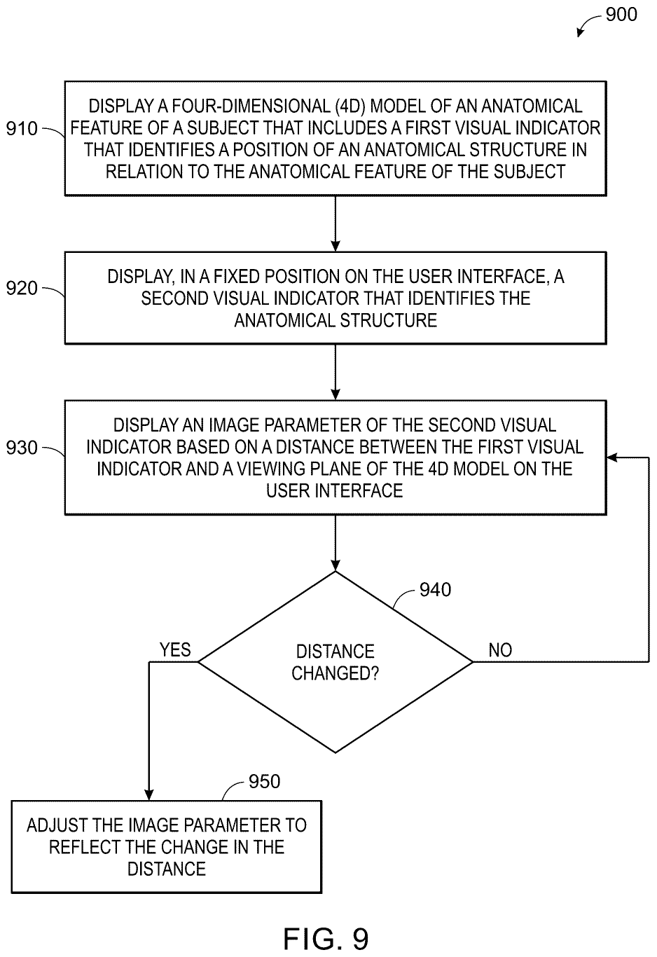

FIG. 9 is a flowchart of an example process for adjusting an image parameter of a second visual indicator that identifies an anatomical structure based on a distance between a first visual indicator, that identifies a position of the anatomical structure relative to an anatomical feature, and a viewing plane of a displayed 4D model. According to an embodiment, the process 900 may be performed by the medical imaging system 110. Alternatively, one or more operations of the process 500 may be performed by another device.

As shown in FIG. 9, the process 900 may include displaying a four-dimensional (4D) model of an anatomical feature of a subject that includes a visual indicator that identifies a position of an anatomical structure in relation to the anatomical feature of the subject (operation 910). For example, the medical imaging system 110 may display a 4D model of an anatomical feature of a subject that includes a visual indicator that identifies a position of an anatomical structure in relation to the anatomical feature of the subject. For instance, the medical imaging system 110 may display the 4D model in a similar manner as described above in connection with operation 540 of FIG. 5.

As further shown in FIG. 9, the process 900 may include displaying, in a fixed position on the user interface, a second visual indicator that identifies the anatomical structure (operation 920). For example, the medical imaging system 110 may display, in a fixed position on the user interface, a second visual indicator that identifies the anatomical structure. The second visual indicator may be an icon, text, a graphic, or the like, that identifies the anatomical structure. The medical imaging system 110 may display the second visual indicator in a fixed position on the user interface. For example, the medical imaging system 110 may display the second visual indicator in a user interface element (e.g., table, legend, chart, etc.) that is displayed in a fixed position on the user interface. In this way, a user can assess what anatomical structures are present in relation to the 4D model based on the respective second visual indicators presented in the user interface element.

As further shown in FIG. 9, the process 900 may include displaying an image parameter of the second visual indicator based on a distance between the first visual indicator and a viewing plane of the 4D model on the user interface (operation 930). For example, the medical imaging system 110 may display an image parameter of the second visual indicator based on a distance between the first visual indicator and a viewing plane of the 4D model on the user interface. The image parameter may be an opacity, a brightness, a resolution, a color, a shading, or the like. The medical imaging system 110 may display the image parameter based on a distance between the first visual indicator and a viewing plane of the 4D model. For example, if the first visual indicator is visible in the viewing plane of the 4D model, then the medical imaging system 110 may display the second visual indicator using an image parameter that designates that the first visual indicator is visible. Further, if the first visual indicator is not visible in the viewing plane of the 4D model, then the medical imaging system 110 may display the second visual indicator using an image parameter that designates that the first visual indicator is not visible in the viewing plane of the 4D model. For instance, the medical imaging system 110 may adjust the opacity, brightness, color, or the like, of the second visual indicator based on whether the first visual indicator is visible in the viewing plane and/or based on how visible the first visual indicator is visible in the viewing plane. Restated, the image parameter may indicate the extent of visibility of the anatomical structure in the viewing plane of the 4D model. That is, an anatomical structure that is highly visible in the viewing plane of the 4D model may have a second visual indicator that is displayed differently than a second visual indicator for an anatomical structure that is less visible, or non-visible, in the viewing plane of the 4D model.

As further shown in FIG. 9, the process 900 may include determining whether the distance between the first visual indicator and a viewing plane of the 4D model has changed (operation 940). For example, the medical imaging system 110 may determine whether the distance between the first visual indicator and the viewing plane of the 4D model has changed.

The medical imaging system 110 may determine whether a user input that manipulates the 4D model is received, and determine whether the distance between the first visual indicator and the viewing plane of the 4D model has changed based on whether a user input is received.

As further shown in FIG. 9, if the distance between the first visual indicator and the viewing plane of the 4D model has not changed (operation 940-NO), then the process 900 may return to operation 930. For example, the medical imaging system 110 may maintain the display of the image parameter of the second visual indicator based on determining that the distance between the first visual indicator and the viewing plane of the 4D model has not changed.

As further shown in FIG. 9, if the distance between the first visual indicator and the viewing plane of the 4D model has changed (operation 940-YES), then the process 900 may include adjusting the image parameter to reflect the change in the distance between the first visual indicator and the viewing plane of the 4D model (operation 950). For example, the medical imaging system 110 may adjust the image parameter to reflect the change in the distance between the first visual indicator and the viewing plane of the 4D model. The medical imaging system 110 may adjust the image parameter by adjusting the opacity, adjusting the brightness, adjusting the color, or the like, of the second visual indicator based on a change in the distance between the first visual indicator and the viewing plane of the 4D model. For example, if the first visual indicator is moved from a first position that is more visible in the viewing plane to a second position that is less visible in the viewing plane, then the medical imaging system 110 may increase an opacity of the second visual indicator, decrease a brightness of the second visual indicator, or the like. In this way, a user can assess that the anatomical structure is being moved outside of the viewing plane based on the change in the image parameter of the second visual indicator. Alternatively, if the first visual indicator is moved from a first position that less visible in the viewing plane to a second position that is more visible in the viewing plane, then the medical imaging system 110 may decrease an opacity of the second visual indicator, increase a brightness of the second visual indicator, or the like. In this way, a user can assess that the anatomical structure is being moved into the viewing plane based on the change in the image parameter of the second visual indicator. As another example, if the first visual indicator is moved from a first position that is visible in the viewing plane to a second position that is not visible in the viewing plane, then the medical imaging system 110 may display the second visual indicator as being entirely opaque, as being a minimum brightness, or the like. In this way, a user can assess that the anatomical structure is not visible within the viewing plane based on the change in the image parameter of the second visual indicator. As another example, if the first visual indicator is moved from a first position that is partially visible in the viewing plane to a second position that is entirely visible in the viewing plane, then the medical imaging system 110 may decrease the opacity, increase the brightness, or the like, of the second visual indicator. In this way, a user may be apprised of whether the first visual indicator is visible in the viewing plane of the 4D model and/or the extent of visibility of the first visual indicator in the viewing plane of the 4D model.

Although FIG. 9 depicts particular operations and a particular sequence of operations, it should be understood that the process 900 may include different operations or differently sequenced operations than as shown in FIG. 9 in other embodiments.

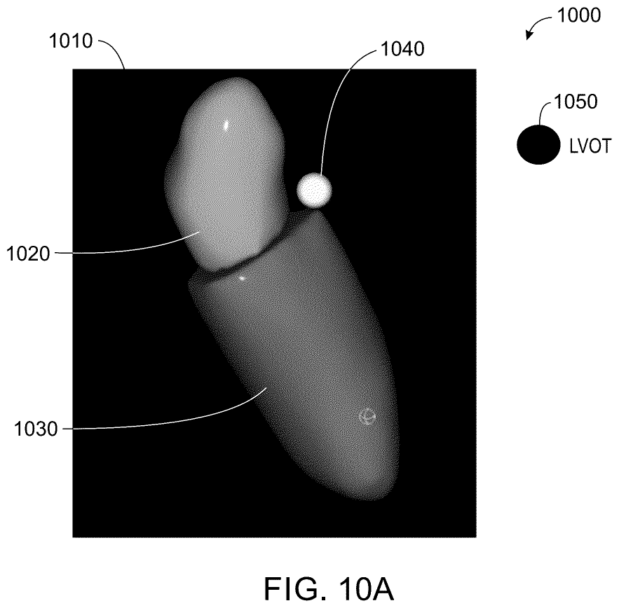

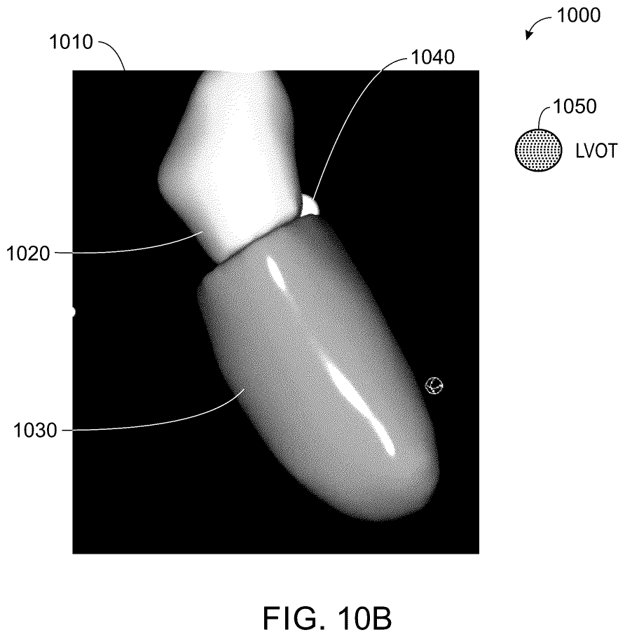

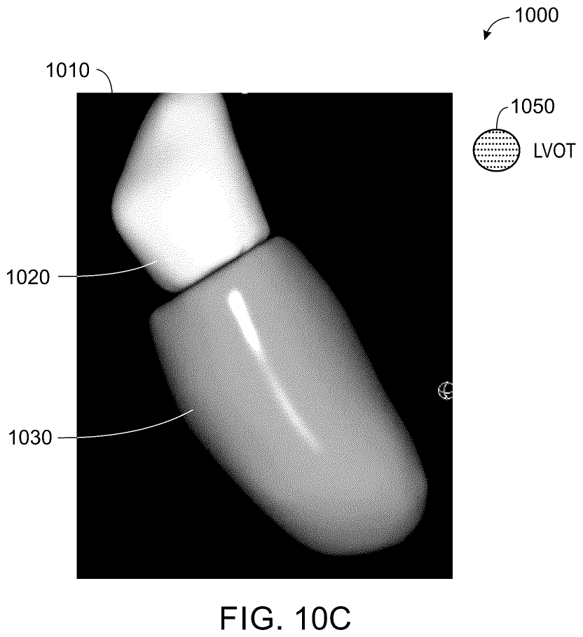

FIGS. 10A-10C are diagrams of an example user interface 1000 for displaying and adjusting an image parameter of a second visual indicator that identifies an anatomical structure based on a distance between a first visual indicator, that identifies a position of the anatomical structure in relation to an anatomical feature, and a viewing plane of a displayed 4D model. As shown in FIG. 10A, the medical imaging system 110 may display a 4D model 1010 that displays the left atrium 1020 and the left ventricle 1030. In this case, the anatomical feature may be the left atrium 1020, the left ventricle 1030, or the left atrium 1020 and the left ventricle 1030. Further, as shown in FIG. 10A, the medical imaging system 110 may display a first visual indicator 1040 that identifies a position of a left ventricular outflow tract. In this case, the anatomical structure may be the left ventricular outflow tract. Further, as shown in FIG. 10A, the medical imaging system 110 may display a second visual indicator 1050 that identifies the anatomical structure. For instance, as shown, the second visual indicator 1050 may include an icon in the form of a circle that is displayed adjacent to text describing the anatomical structure. Further, as shown, the medical imaging system 110 may display the second visual indicator in a fixed position on the user interface 1000. Although a single first visual indicator and a single second visual indicator are shown, it should be understood that, in other embodiments, multiple first visual indicators and multiple corresponding second visual indicators may be shown. As shown in FIG. 10A, the medical imaging system 110 may display the second visual indicator as a non-opaque icon based on a distance between the first visual indicator and a viewing plane of the 4D model 1010. In other words, the first visual indicator is generally entirely visible in the viewing plane of FIG. 10A. Accordingly, the medical imaging system 110 may display the second visual indicator 1050 to reflect that the first visual indicator is generally entirely visible.

As shown in FIG. 10B, the medical imaging system 110 may adjust the display of the 4D model 1010 based on a user input that manipulates the 4D model 1010. For instance, as shown, the medical imaging system 110 may rotate the 4D model 1010 such that the first visual indicator 1040 is less visible than as compared to the displayed 4D model 1010 in FIG. 10A. In this case, the medical imaging system 110 may adjust the display of the image parameter of the second visual indicator 1050 to reflect a change in a distance between the first visual indicator 1040 and a viewing plane of the 4D model 1010. For example, as shown, the medical imaging system 110 may adjust the display of the image parameter of the second visual indicator 1050 such that the second visual indicator 1050 is more opaque than as compared to FIG. 10A.

As shown in FIG. 10C, the medical imaging system 110 may further adjust the display of the 4D model 1010 based on a user input that manipulates the 4D model 1010. For instance, as shown, the medical imaging system 110 may rotate the 4D model 1010 such that the first visual indicator 1040 is less visible than as compared to the displayed 4D model 1010 in FIGS. 10A and 10B. Restated, the first visual indicator 1040 is not visible in the viewing plane of the 4D model 1010 in FIG. 10C. In this case, the medical imaging system 110 may adjust the display of the image parameter of the second visual indicator 1050 to reflect a change in a distance between the first visual indicator 1040 and a viewing plane of the 4D model 1010. For example, as shown, the medical imaging system 110 may adjust the display of the image parameter of the second visual indicator 1050 such that the second visual indicator 1050 is more opaque than as compared to FIG. 10A and FIG. 10B.

In effect, the medical imaging system 110 may, in real-time, adjust the image parameter of the second visual indicator 1050 as the user manipulates the 4D model 1010. For instance, as the 4D model 1010 is rotated such that the first visual indicator 1040 is brought out of view, the medical imaging system 110 may fade the second visual indicator 1050 to indicate that the first visual indicator 1040 is being brought outside of view.

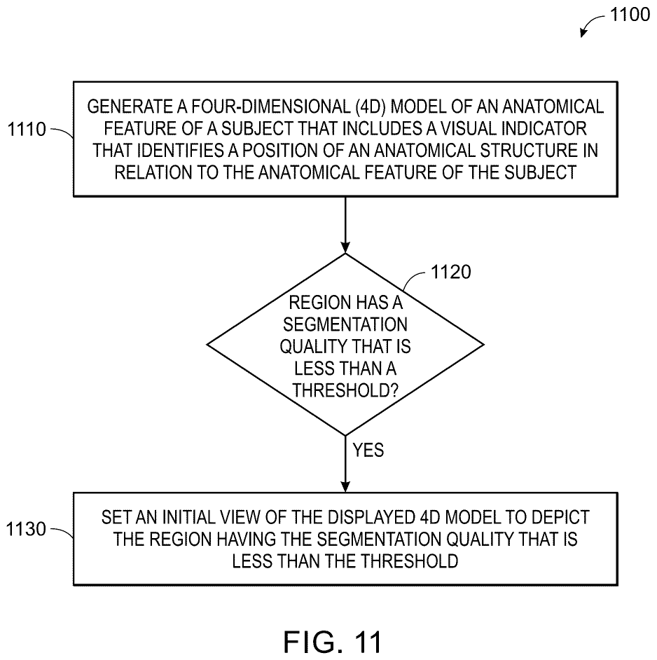

FIG. 11 is flowchart of an example process 1100 for setting an initial view of a displayed 4D model to depict a region of the displayed 4D model having a segmentation quality that is less than a threshold. According to an embodiment, the process 1100 may be performed by the medical imaging system 110. Alternatively, one or more operations of the process 1100 may be performed by another device.

As shown in FIG. 11, the process 1100 may include generating a four-dimensional (4D) model of an anatomical feature of a subject that includes a visual indicator that identifies a position of an anatomical structure in relation to the anatomical feature of the subject (operation 1110). For example, the medical imaging system 110 may generate a 4D model of the anatomical feature of the subject that includes a visual indicator that identifies the position of the anatomical structure in relation to the anatomical feature of the subject in a similar manner as described above in connection with operation 530 of FIG. 5.

As further shown in FIG. 11, the process 1100 may include determining whether a region of the 4D model has a segmentation quality that is less than a threshold (operation 1120). For example, the medical imaging system 110 may determine whether a region of the 4D model has a segmentation quality that is less than a threshold. The medical imaging system 110 may determine a segmentation quality of one or more regions of the 4D model. For example, the medical imaging system 110 may determine a segmentation quality based on a comparison between the segmented anatomical feature and a model, based on performing an AI technique, based on an output of a segmentation algorithm, based on a distance between a visual indicator for an anatomical structure and the anatomical feature of the 4D model, or the like. The medical imaging system 110 may determine respective segmentation qualities for one or more regions of the 4D model. For example, the medical imaging system 110 may determine segmentation qualities for a predetermined number of regions of the 4D model. Alternatively, the medical imaging system 110 may determine segmentation qualities of regions of the 4D model that are adjacent to visual indicators associated with anatomical structures.

As further shown in FIG. 11, if the region of the 4D model has the segmentation quality that is less than the threshold, then the process 1100 may include setting an initial view of the displayed 4D model to depict the region having the segmentation quality that is less than the threshold (operation 1130). For example, the medical imaging system 110 may set an initial view of the displayed 4D model to depict the region having the segmentation quality that is less than the threshold. According to an embodiment, the medical imaging system 110 may highlight the region having the segmentation quality that is less than the threshold. For example, the medical imaging system 110 may adjust an image parameter of the region, may include a bounding box around the region, may display an indication that the region has the segmentation quality that is less than the threshold, or the like. In this way, a user can be apprised of a particular region of the 4D model that might not be accurate, might need to be revised, or the like.

Although FIG. 11 depicts particular operations and a particular sequence of operations, it should be understood that the process 1100 may include different operations or differently sequenced operations than as shown in FIG. 11 in other embodiments.

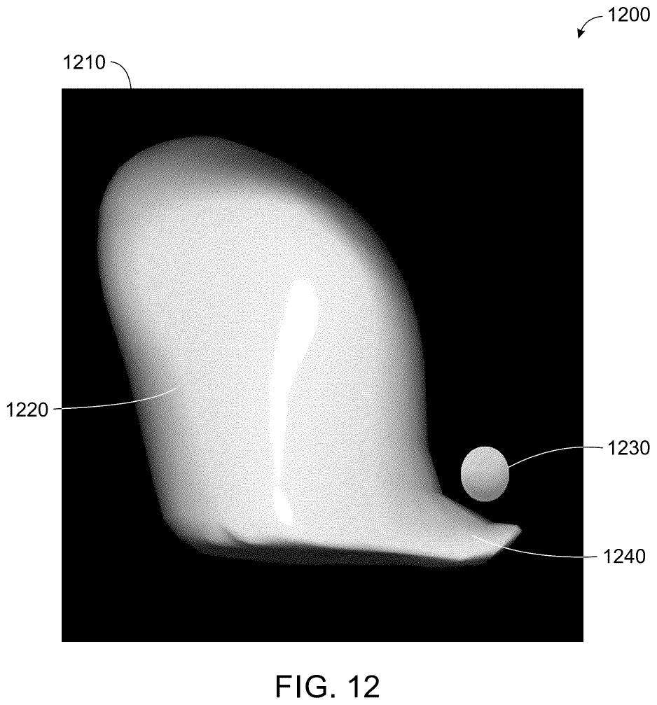

FIG. 12 is a diagram of an example user interface for displaying an initial view of a 4D model to depict a region of the displayed 4D model having a segmentation quality that is less than a threshold. As shown in FIG. 12A, the medical imaging system 110 may display a 4D model 1210 that displays the left atrium 1220. In this case, the anatomical feature may be the left atrium 1220. Further, as shown in FIG. 12A, the medical imaging system 110 may display a visual indicator 1230 that identifies a position of a left atrial appendage. In this case, the anatomical structure may be the left atrial appendage. As further shown in FIG. 12A, the medical imaging system 110 may display a region 1040 that is associated with a segmentation quality that is less than a threshold. For instance, as shown, the region 1040 may correspond to a confluence of the pulmonary veins and the left atrial appendage. The region 1040 may be a region that should not have been delineated as a border of the left atrium 1220.

According to an embodiment, the medical imaging system 110 may acquire medical imaging data of an anatomical feature of a subject; determine a position of an anatomical structure in relation to the anatomical feature of the subject; generate a four-dimensional (4D) model of the anatomical feature of the subject that includes a visual indicator that identifies the position of the anatomical structure in relation to the anatomical feature of the subject; and display the 4D model via a user interface.

According to another embodiment, the medical imaging system 110 may acquire medical imaging data of one or more anatomical features of a subject; determine one or more positions of one or more anatomical structures in relation to one or more anatomical features of the subject; generate a four-dimensional (4D) model of the one or more anatomical features of the subject that includes one or more visual indicators that identify the one or more positions of the one or more anatomical structures in relation to the one or more anatomical features of the subject; and display the 4D model via a user interface.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. An ultrasound imaging system comprising:
a transducer configured to transmit and receive ultrasound signals;
a matching layer configured to have an acoustic impedance between a tissue to be imaged and a material of the transducer;
a damping block configured to absorb ultrasound energy; and
a processing circuit configured to:
acquire medical imaging data of an anatomical feature of a subject;
determine a position of an anatomical structure in relation to the anatomical feature of the subject;
generate a four-dimensional (4D) model of the anatomical feature of the subject that includes a visual indicator that identifies the position of the anatomical structure in relation to the anatomical feature of the subject; and
display the 4D model via a user interface.

2. The ultrasound imaging system of claim 1, wherein the one or more processors are further configured to:
receive a user input that manipulates the displayed 4D model; and
adjust the displayed 4D model based on the user input while maintaining a position of the visual indicator relative to the displayed 4D model.

3. The ultrasound imaging system of claim 1, wherein the visual indicator is a first visual indicator, and wherein the one or more processors are further configured to:
display, in a fixed position on the user interface, a second visual indicator that identifies the anatomical feature; and
adjust an image parameter of the second visual indicator based on a distance between the first visual indicator and a viewing plane of the 4D model on the user interface.

4. The ultrasound imaging system of claim 3, wherein the image parameter is a brightness of the second visual indicator, an opacity of the second visual indicator, or a color of the second visual indicator.

5. The ultrasound imaging system of claim 1, wherein the one or more processors are further configured to:
set an initial view of the displayed 4D model to depict a region having a segmentation quality that is less than a threshold.

6. The ultrasound imaging system of claim 1, wherein the one or more processors are further configured to:
segment the anatomical feature in the medical imaging data.

7. The ultrasound imaging system of claim 1, wherein the one or more processors are further configured to:
determine a region of the 4D model that is associated with a segmentation quality that is less than a threshold; and
set an initial view of the 4D model to depict the region.

8. A method comprising:
acquiring medical imaging data of an anatomical feature of a subject;
determining a position of an anatomical structure in relation to the anatomical feature of the subject;
generating a four-dimensional (4D) model of the anatomical feature of the subject that includes a visual indicator that identifies the position of the anatomical structure in relation to the anatomical feature of the subject; and
displaying the 4D model via a user interface.

9. The method of claim 8, further comprising:
receiving a user input that manipulates the displayed 4D model; and
adjusting the displayed 4D model based on the user input while maintaining a position of the visual indicator relative to the displayed 4D model.

10. The method of claim 8, wherein the visual indicator is a first visual indicator, and wherein the method further comprises:
displaying, in a fixed position on the user interface, a second visual indicator that identifies the anatomical feature; and
adjusting an image parameter of the second visual indicator based on a distance between the first visual indicator and a viewing plane of the 4D model on the user interface.

11. The method of claim 10, wherein the image parameter is a brightness of the second visual indicator, an opacity of the second visual indicator, or a color of the second visual indicator.

12. The method of claim 8, further comprising:
setting an initial view of the displayed 4D model to depict a region having a segmentation quality that is less than a threshold.

13. The method of claim 8, further comprising:
segmenting the anatomical feature in the medical imaging data.

14. The method of claim 8, further comprising:
determining a region of the 4D model that is associated with a segmentation quality that is less than a threshold; and
setting an initial view of the 4D model to depict the region.

15. A non-transitory computer-readable medium storing instructions that, when executed by one or more processors, cause the one or more processors to:
acquire medical imaging data of an anatomical feature of a subject;
determine a position of an anatomical structure in relation to the anatomical feature of the subject;
generate a four-dimensional (4D) model of the anatomical feature of the subject that includes a visual indicator that identifies the position of the anatomical structure in relation to the anatomical feature of the subject; and
display the 4D model via a user interface.

16. The non-transitory computer-readable medium of claim 15, wherein the instructions further cause the one or more processors to:
receive a user input that manipulates the displayed 4D model; and
adjust the displayed 4D model based on the user input while maintaining a position of the visual indicator relative to the displayed 4D model.

17. The non-transitory computer-readable medium of claim 15, wherein the visual indicator is a first visual indicator, and wherein the instructions further cause the one or more processors to:
display, in a fixed position on the user interface, a second visual indicator that identifies the anatomical feature; and
adjust an image parameter of the second visual indicator based on a distance between the first visual indicator and a viewing plane of the 4D model on the user interface.

18. The non-transitory computer-readable medium of claim 17, wherein the image parameter is a brightness of the second visual indicator, an opacity of the second visual indicator, or a color of the second visual indicator.

19. The non-transitory computer-readable medium of claim 15, wherein the instructions further cause the one or more processors to:
set an initial view of the displayed 4D model to depict a region having a segmentation quality that is less than a threshold.

20. The non-transitory computer-readable medium of claim 15, wherein the instructions further cause the one or more processors to:
segment the anatomical feature in the medical imaging data.
